(19) United States
(12) Patent Application Publication
(10) Pub. No.: US 2006/0216364 A1
(43) Pub. Date: Sep. 28, 2006

(54) Composition for Topic Use Containing an Extract of Stryphnodendron...

(75) Inventors: Suzelei de Castro Franca, Ribeirao Preto/SP (BR); Joao Carlos Nuncs de Oliveira, Ribeirao Preto/SP (BR); Lunz Pasqualin, Ribeirao Preto/SP (BR); Lucelio Bernades Couto, Ribeirao Preto/SP (BR); Raphael Carlos Comelli Lia, Araraquara (BR)

Correspondence Address: SMITH HOPEN, PA, 180 PINE AVENUE NORTH, OLDSMAR, FL, 34677, US

(73) Assignees: Aerp Associacao de Ensino de Ribeirao Preto, Ribeirao Preto/SP (BR); Apsen Farmaceutica S.A., Ribeirao Preto/SP (BR)

(21) Appl. No.: 11/381,655

(22) Filed: May 4, 2006

Related U.S. Application Data
(63) Continuation of application No. PCT/BR04/00221, filed on Nov. 11, 2004.

(51) Int. Cl. A61K 36/70

(52) U.S. Cl. 424/725

(57) ABSTRACT

The present invention relates to topic compositions containing an extracts of the plants of genus Stryphnodendron, their preparation as well as their application. The compositions contain 1-6 % of total phenols and are useful for the treatment of cutaneous wounds.

### TECHNICAL FIELD

The present invention belongs to the field of pharmaceutical segment, particularly consisting of a phytotherapeutic pharmaceutical formula for the treatment of ulcers, either those ones that obstruct or decrease blood irrigation, and more particularly pressure ulcers and varicose ulcers (both venous or arterial), besides the preparation process and its application, being said phytotherapeutic pharmaceutical forms featured for the reason of having gross extract and portions of plants of the gender Stryphnodendron.

### FUNDAMENTALS OF THE TECHNIC

As it is of general knowledge, from thousands of years wounds have been treated aiming at enhancing the natural scar healing process. It is known that some wounds will become scar healed by themselves whilst others will resist from any efforts to do so becoming some of these wounds cases that will lead persons to diabetes, cancer, varicose ulcers and pressure ulcers originated from a long period of time lying in bed, best known as crusts.

In accordance with Modolin and Bevilacqua (1985) the scar healing is a physiological process that begins through an inflammatory response featured by an increase on the blood flow, capillary permeability and migration of leococitos to the harmed region. The capillary permeability provides the extravasation of plasma and its components thus forming the inflammatory waste material.

At the start a wound is filled by clots, insoluble whitish proteins and waste material that form up a crust that isolates the wound from the external side almost at once (COTRAN, 1989). The neutrophils and macrophages are the first cells to migrate into the wounded region in a response from the organism against the bacterial invasion (RUNNELS et. al., 1976) and when the cells destroy themselves and waste away the bacteria degenerate themselves forming up the pus with the dead remaining tissue (GUYTON, 1991).

When the granulation tissue contracts itself it retracts the edges of the wound in the skin towards the central part of the wound thus allowing the area to be reformed and become smaller. When granulation is excessive a delay occurs to the scar healing. Prevention can be obtained by encouraging a subcrustal scar healing when the formation of a crust wound encourages the scar healing process. The crust appearance can occur by the use of several substances as the tannins. When the wound waste material lingers in the desaggregation on the crust occurs, thus allowing the development of germs between the wound and the granulation tissue (OLIVEIRA 1992).

As it is of general knowledge, pressure ulcers or crusts are excavated lesions that might reach variable depths (named “grades”) reaching the skin and as well even the muscles and bones, being said crusts caused by a closure in the blood circulation as a consequence of prolonged pressure on the bones of the musculatura and on the neighboring region. The obstruction blocking on the blood circulation results from lack of normal movements of the patient in addition to the deficit of sensitivity that are the basic reasons of this condition that shows necrosis areas that further develops into the appearance of wounds.

The crusts take place in several parts of the body and in most cases on the salient bones that forces up the soft tissues of the patient against hard surfaces that encourages the lesion to appear. For instance, a patient who is lying in bed for a long time exercises pressure on his/her heel through the weight of his/her leg thus forcing a small area of the skin and this pressure is sufficient to block the blood vessels between the bone of the heel and the skin of the surface so forming up a crust ulcer on the heel. In his/her turn, the patient who stays for a long time lying on his/her back not ever moving himself/herself will exercise pressure on the sacro bone thus allowing the crust to originate from there, being this one the place where the crusts are most usually found in. Other regions as the clavicules, elbows, ribs and occipital bones are also very subject to the appearance of crusts.

Therefore, the appearance of crusts begins with the prolonged pressure on determined part of the body up to the time when the wound is originated by a lesion of surrounding skin.

In their turn, the varicose ulcers (venous or arteriais ones) are a consequence from a backlog of venous blood in the skin that determines their thickness, the reduction of subcutaneous fat lard and brown stains. In the arterial hypertension, diabetes and arterial trombose the obstrution of the skin arteries or of the profound tissues occurs, thus originating the wound. As for the infectious ulcers they are painful and show inflammatory characteristic and have pus secretion.

The ulcers (pressure or varicose ones) may be described in accordance with the characteristic stages of tissue loss usually classified into grades in accordance with the standards of the Quick Reference Guide for Clinicians, being these grades:
Grade I—skins shows hypermia (redness)
Grade II—the wound penetrates the skin (epidermis and/or “dermis) causing also damage or necrosis in the subcutaneous tissue. The lesion clinically appears as a superficial surface ulcer with or without damage to the adjacent tissue.
Grade III—loss of skin and tissues that extends to the subcutaneous tissues; and
Grade IV—the lesion reaches the muscle articular and bone tissues.

The damaged tissue with waste material is a fiber optic tissue mostly composed of non-organized colagen fibers. This type of tissue is of little vascularity, of little nervure and unstretchable. Owing to the absence of nervure and loss of elasticity it is in a fast way susceptible to a series of injuries. The injured tissue is hard to scar healing due to the poor local vascularity.

Numerous types of treatment are used for pressure ulcers that involve preventing measures and several types of therapies that might be of type: a) local treatment or care using special beds, water mattress, etc; b) patient treatment that includes asepsis, nutrition improvement, care and fight against local infections, etc; and c) use of physical therapies over the affected area with electric stimulation, ultrasound, laser therapy and others.

In a general manner the scar healing process that naturally takes place in response to the injury includes the inflammatory response, angiogenese, synthesis and deposition of colagen and formation. Although tissue repair and regeneration occur without an intervention, scar healing as a second intention might occur due to the acceleration of the colagen synthesis. Thus, the scar healing of wounds involves a series of complex biochemical and cellular events which finally promote a retraction, closing and scar healing of the lesion.

The term “revitalization” is used as a means to restore the vascularity and elasticity of the tissue that had been injured and crusted. The term “injury” is used as a method of provoking the wound caused by surgery, traumas, ulcers, burns, chemical agents and virus or bacteria infections. The term “crusted” tissue is a type of fiber optical tissue or colagen formed during the scar healing of the wound or other pathological processes. The crusted scarred tissue is a fiber optical tissue produced by hundreds of non-organized colagen fibers and is formed by injuries or inflammation in tissue site.

The contamination of a wound may be a result from the direct contact with the infected object or by an incoming dirty, dust or exogeneous microorganisms from the patient's skin or from the gastric-intestinal causes. For instance, it has been recognized that despite of adopted effective measures to avoid infections, practically all burns start up a colony of bacteria within a period of 12-24 hours.

In a general manner, infection bars the scar healing of the wound due to the provokation of tissue damage and as well due to the inflammation. Subsequently, the recovery of the wound is damaged by a progressive inflammation, by formation of waste materials, by release and activation of enzymes, by generation of free radicals, consumption of oxygen and the loss of nervure. Therefore, cares to prevent inflammation might lead to the scar healing of the wound not risking the ability of the tissue to resist the infection and to inhibit the essential function of the macrophages.

The prior art teaches that the medical protocols utilized for scar healing of wounds are very much varied, having such proceedings their benefits and limitations as well. The actives available for topic therapeutics include the use of: antibiotics; bismuth salts; carbohydrates; hormones; plasma; zinc and tannic acid; treatments with electric stimulation; hyperbaric oxygenation; ultrasonic therapy and laser.

Still taking into consideration the traditional protocols the wounds treatment has been intensified in relation to the use of natural products to aid scar healing, such as copaiba oil (Coorea 1984;
Eurides & Mazzanti, 1995); papaina (Sanchez Neto et al., 1993); sugar (Prata et al., 1988); colagen (Abramo, 1990) and vitamin A (Bondi, 1989).

The Barbatiman (stryphnodendron po/yphy//um and S. adstringens; Martius) , particularly Ihe phytotherapic ones dealt with in this development of innovated pharmaceutical formulation, comprise a leguminous mimosoidea arboreal from the Brazilian flora that is found from the State of Para up to the State of Sao Paulo. The barks are thick and have binding effect related to the presence of active principles like taninns.

It may be used indistinctively extracts from the barks of these species in the preparation of scar healing phytotherapeutics owing the similarity of chemical composition.

It was mentioned by the Brazilian Pharmacology (1959) that the barks from the plant have tannins that hold important pharmacological activities on mice like anti- inflammatory actions (LIMA, 1998) and scar healing in dermis wound (PANIZZA, 1998 and EURIDES, 1996), in gastric ulcer (FAVARETTO et al., 1985) and duodenal (RIZZINI & MORS, 1976). Taninns also assist in the treatment of eczema with the advantage of not generating collateral effects usually observed in Ihe therapies by using high dosages of glycocorticoids (MROWIETS et al., 1991).

In accordance with Mello (1998) the main features of the extracts made from the barks of Barbatiman is found in the richness of taninns. In '1996 Mello et al. (apud MELLO, 1998) isolated and identified 22 compounds on gross extract of barks from Barbatiman, being all of them taninns of condensed type, being this an active principles involved in several biological activities and the one that makes of 25-30% of rhe plant bark (PANIZZA et al., 1998). Proantocianidinas, prorobinetinidinas, flavan-3-01 and prodelfinidinas were the chemical portions studied. According to Lima et al. (1998) The anti-inflammatory activity of Barbatiman is attributed to the presence of proantocianidinas.

Other studies have investigated the composition of taninns in three species of Barbatiman, that is, The S. polyphy//um, The S. adstringens and The Dimorphandra mo//is (SANTOS, 2002) wherein the same were already evaluated chemically. It is to be emphasized that the D. Mo//is is known as Barbatiman, nevertheless it differs chemically from the two aforesaid specimens as it is shown below on Table

Cromatographic Analysis of Barbatiman Extract after Hidrolise

| | | | | Galic | |
|---|---|---|---|---|---|
| Species | Plant | Delphinidina | Cyanidina | Acid | Flavonols// |
| S. adstringens | Bark | + | − | + | − |
| | Leaf | + | − | + | + |
| S. polyphyllum | Bark | + | − | + | − |
| | Leaf | + | − | + | + |
| D. mollis | Bark | − | + | − | + |
| | Leaf | − | − | − | + |

In accordance with Haslam (1996) taninns combine themselves with the proteins through chemically well defined links chains which provide the binding feature. This complex tannin/protein and/or polysacharideo forms a protecting coating on the skin or on the mucosa damaged. Under this coating the natural process of recovering the wounds, the bums and the inflammations might occur.

Tannins also hold inhibiting effects on bacteria and fungi (antimicrobial activity), being it based on the assumptions that the tannins Inhibit the enzymes of the bacteria and fungi acting with the substrates of these enzymes. Furthermore, tannins act on the cellular membranes of microorganisms changing their metabolism and complexion of taninns with metallic ions decreasing the availability of essential ions for the metabolism of the microorganisms (SIMOES, 2000).

Neto et al. (1996) compare the scar heating action of Calendula officinalis L and Stryphnodendron barbatiman (Vellozo) Martius (Barbatiman) in the treatment of varicose ulcers and skin lesions in humans. The results show that treatments whatever with calendula and as well with barbatiman are efficient in the recovery of burns lesions and in varicose ulcers once the association allowed a faster scar healing.

Earlier studies disclose that one of physiotheraphics alternatives for accelerating the tissue process is the use of laser. The laser is a special form of electromagnetic energy that operates in the region of visible electromagnetic specter or infrared that classifies many different types of laser, whichever whatever high or low power lasers.

In accordance with Baxter et al. (1994) the success with the use of laser is due to the responses induced in the tissues such as the reduction of edema, decrease in the inflammatory process, increase in the fagocitose process, colegen synthesis of colagen and of re-formation, being the most of registered biological effects related to the proliferation of cells involved in the scar healing, mainly those of fiber blasts (f and macrophages (O'KANE et al., 1994).

In vitro studies are reported more frequently in the reduction of scar healing time of wounds in the cutaneous and mucosa extracts particularly on the proliferation and activation of the protein synthesis of fiber blasts, the principal cell responsible for the tissue restoration (ABERGEL, 1984; BOUL TON, 1986; HALLMAN, 1988; LOEVSCALL & ARENHOLT-BINDSLEV,1994).

Laser is a non-invasive instrument important in the recovery of ulcers (SCHINDL et al., 1999) and of numerous etiologies, including venous and diabete ulcers (ALGAN et al. 2001) being the scar healing time dependent on size and cause of ulcers.

In 1991 Arantes disclosed that the application of low intensity laser in dermis lesions of lower limbs induced to cures without comeback. In 70% of patients and that 30% had improved diagnostic when compared to normal clinical proceedings through which a cure of 38% was disclosed, an improvement of 48% was disclosed and a 14% of absence of any recovery was disclosed.

Besides the treatment with topic products for scar healing, the physiotherapeutic appatuses as the ultrasound (US) and low intensity laser serve as alternatives to the treatment in tissue recovery.

The ultrasound comprises another method widely used to accelerate tissue recovery process, being its functioning due to thermal and non-thermal effects (mechanical) generated by the ultrasonic waves on the damaged tissues (STARCKEY, 2001; DYSON, 1992). The therapeutic effects carried out by the low intensity US are usually due to the non-thermal effects produced by stable waves, acoustic currents, micro currents and cavitaties (DYSON, 1987).

The skin, in addition to functions as a barrier, is also a target for the via of drug administration with the advantage of decreasing possible adverse effects caused by oral means of administration or other ones which are considered invasive (MA TCHED, 2002). Nowadays there is an increasing interest about this technique for it allows the transdermal transport of drugs with low molecular weight or even of proteins with a high molecular weight as the insulin which could be avoided being administered in the form of injections thus preventing the accurance of ache and possible permanent damages to the skin.

Recent studies have been carried out with the purpose of altering the cutaneous permeability, including the use of chemical “facilities” (MORGANTI et al., 2001) and the physical “facilities” like iontoforese, an electrotherapeutical technique that utilizes the galvanic current to facilitate the intercutaneous penetration of therapeutic substances (BARRY, 2001) and, of a widespread use, the fonoforese or sonoforese (MATCHED & BOUCAUD, 2002).

This technique is comprised of the association of ultrasound with several drugs used as a means of coupling in the form of gel or ointments changing its diffusion acrossr the comeous extract. The US increases the coefficient of diffusion of the corneous extract to hidrofobics molecules of low and high molecular weight (MITRAGOTRI, 2001; JOSHI & RAJE, 2002), being able such corneous extract of being applied together with chemical facilities (TEZEL et al., 2002) enhancing the absorption of several medicines. In this aspect Skanem & Fentner (1984) disclosed that the ultrasound was employed in different previous works for offering localized conditions in the skin which favored the diffusion of drugs.

Thus, the ultrasonic heating, observed in an insignificant magnitude is responsible for a modest alteration in the intensity, frequence and ways of drugs transportation. The grade of generated heat is considerably influenced by subtle factors like the movement of the transdutor, the anatomical site of application, the quantity and type of vehicle or means of coupling pressures and radiations, the forces generated by the micro currents and the formation of steam or gas bubbles in liquid.

Even before the studies carried out and described in literature up to then, it is noticed that the diffused and popular use of Barbatiman, with or without association of Calendula plant, is still of empirical procedure, that is, for its application in site an infusion of Barbatiman's bark is made, then proceeding on the direct application on the wound, this resulting in a methodology without any kind of penetration control, local moisture, contents of contamination of added agents, etc, being totally lack of any pharmaceutical forms that enable to measure dosages, control and clinical methodologies.

It is concluded this way that this type of procedure could not under no circumstance be repeated in those patients who hold rebel wounds like the type of all ulcers, varicose or pressure ones, once that care about the local asepsis, the prevention against inflammations and infections are the most important topics during the scar healing treatment of wounds for it is necessary the said wounds produce the tissue recovery and regerreration with intervention, that is, the scar healing is meant as second intention, occurring due to the acceleration of the colagen synthesis.

Aiming at creating means of allowing the use of Stryphnodendron barks's properties (Barbatiman) in a pharmacological way in humans and as well with veterinary application, the patent applicant developed the present invention that relates to phytotherapeutical pharmaceutical form for scar healing, its process of preparation, application, active and derived composition, besides demonstrating the treatment with the formulated product wherein extracts and portions of a medicinal plant native from Brazil were validated, particularly the Stryphnodendron polyphythum or Barbatiman with a high content of tannin constituents and total phenols developing in portions of dye and powder with ideal physical and physical—chemical features to receive ingredients and vehicles able of being dosed clinically and as well as pharmaceutically.

The great advantage of using formulations that contain extracts of Stryphnodendron is related to the scar healing power which is enhanced by the antimicrobial action which keep lesioned sites with a minimized level of microorganism thus favoring the scar healing.

The described process was carried out from dyes that were extracted from the Stryphnodendronrs barks, concentrated on rotovaporator and dried by forced circulation of air being such barks subject to quantitative analysis for determination of contituents' contents predominantly tannin and total phenols (AOAC, F. Bras.lV supplement 35, pages 30-2). The average amount of total phenols vary between 40-50%. Following, the preparation of pharmaceuticals forms were carried out with a quality control obeying specifications described in the 4th Edition of Brazilian Pharmacopy.

The, pharmaceutical form was made of different concentrations of actives and through pre-clinical studies. The highest efficiency concentration was verified in the process of scar healing. 150 lesions were evaluated from which 60% had scarred completely.

Literature has disclosed that the scar healing process is affected by the presence of microorganisms, most of them multiresistant.

In vitro microbiological assays using standard especimens and field species isolated from infected crusts made evidence that the Sryphnodendronr's extracts hold GIM 62,5 I1 g/mL for positive grams and 250 I1glmL for negative. The tested pharmaceutical forms contain concentrations of active substances 400 and 100 times to GIM of positive and negative gram respectively.

Clinically it was observed that the infected crusts also responded to the treatment with the active and scar healed without the need of complementary use of antibiotics.

During the accompaniment of clinical study of Barbatiman's physiotherapeutical pharmaceutical form use in accordance with the developed formulation and now protected, the patients—under treatment were subject to macroscopic and microscopical examinations of organs (stomach, kidneys, liver and spleen) besides laboratory examinations like hemograms; biochemical assays of renal and hepatic function before, during and after treatment and the results proved that the phytotherapeutical did not effect the biochemical and hematological parameters analyzed (see graphics) related to figures itemized in continuation.

The results of therapeutical efficiency that were obtained acknowledge the patent application of the phytotherapeutical pharmaceutical form by this way applied for, that is, the form of the phytoderived medicine constituted of 3% of Stryphnodendron's active, in the way that it will be defined further on, being said medicine used in the Clinical Study of Scar Healing Action's Evaluation of 150 crusts with different grades of tissue damage, disclosing the result as follows:
Treated lesions were predominantly of Grade 1 (61.2%) and Grade 11 (20.1%), being the medicine efficient in the complete scar healing of 58.8% for the lesion of Grade I and 35.6% for the lesions of Grade II.
The evolution in the scar healing process showed that the tested phytotherapic provided the scar healing of over 70% of treated lesions within a maximum period Of 2 months.
The time for scar healing varied in accordance to the grade and site of lesion, once the crust of Grade I located in the hips and sacro got scar healed in a period less than one month.
Product was found effective in lesions of all grades

### BRIEF DESCRIPTION OF THE DRAWINGS

In order to complement the present description in a way to obtain a better understanding of the characteristics of the present invention and in accordance with a preferable practical accomplishment of the same, a set of drawings accompanies the attached description wherein, by an exemplified way, although not limiting, the following was disclosed:

FIG. 1 shows a graphic of distribution of patients by range of age that were treated with the innovated product in the form of a spray;

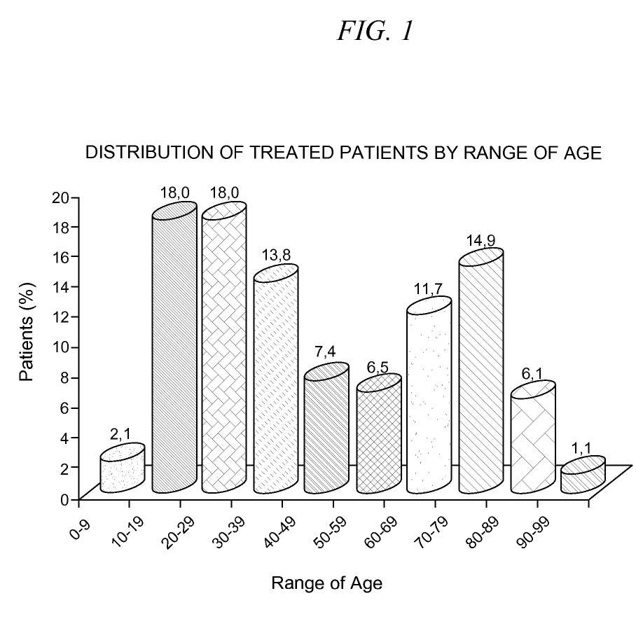

FIG. 2 shows a table graphic of variation in the number of lesions (pressure ulcers) by treated patients;

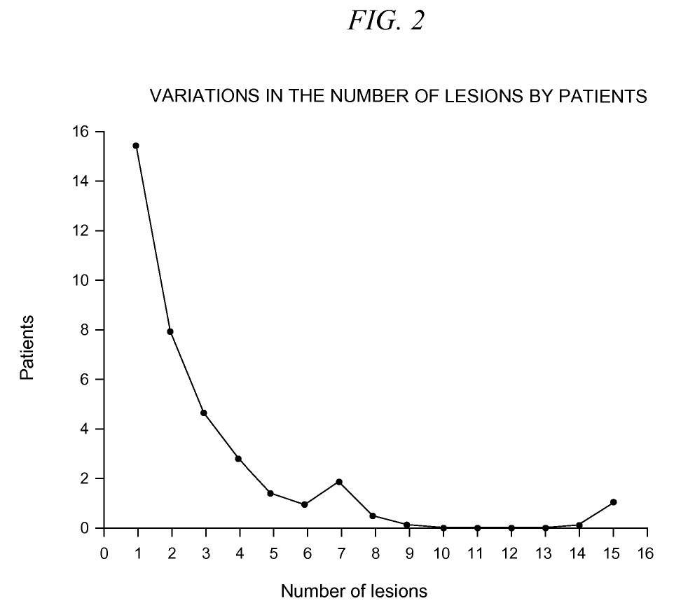

FIG. 3 shows a graphic of “Treated lesions Versus Treatment Period”, disclosing an evolution in scar healing according to the grade of lesions;

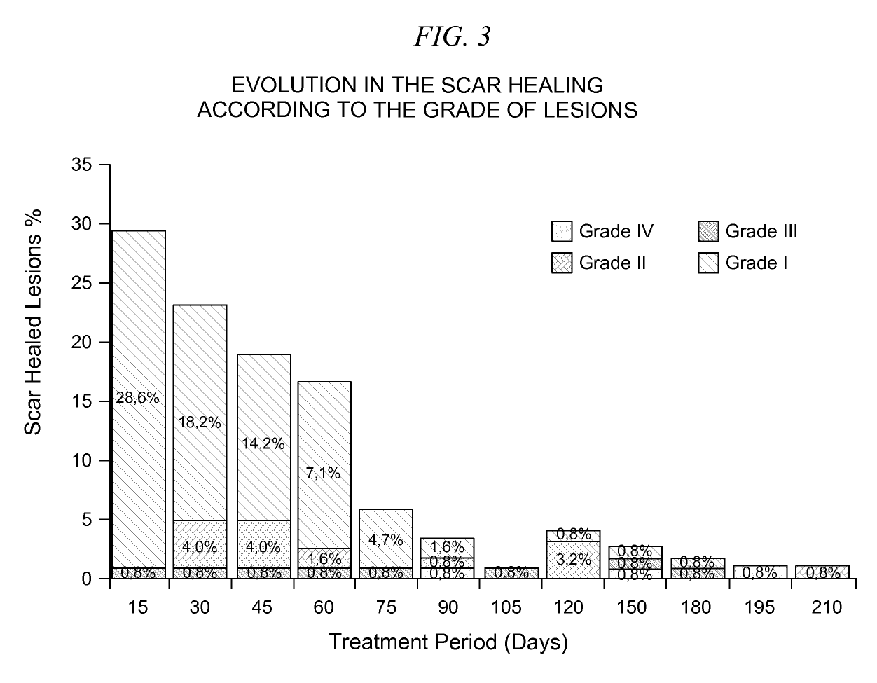

FIG. 4 shows another graphic related to “Treated lesions Versus Treatment Period” indicating the variation in the treatment period by site of lesion occurrence;

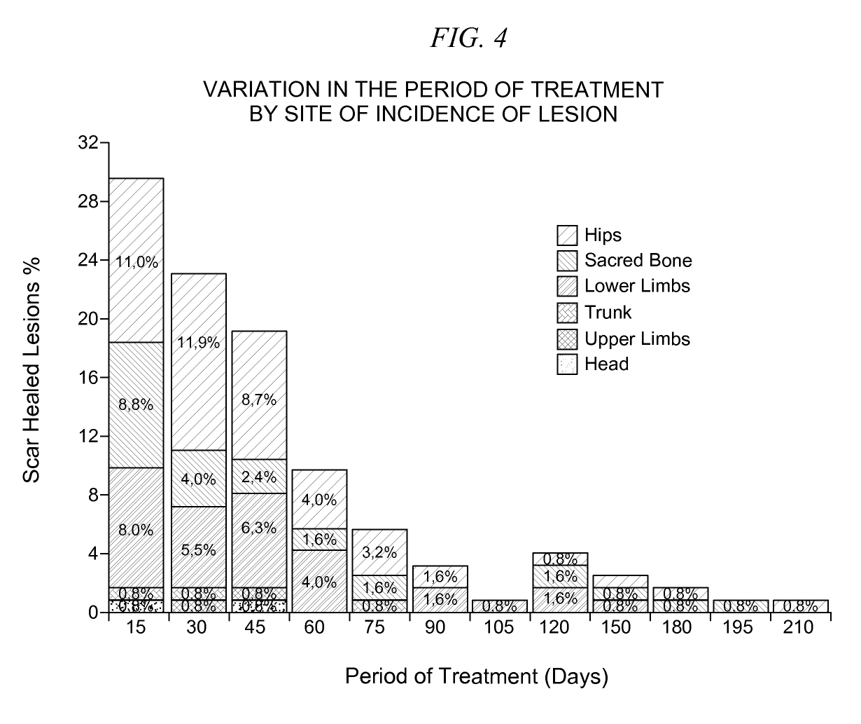

FIG. 5 shows the graphic of distribution of treated lesions (pressure ulcers) by grade of classification;

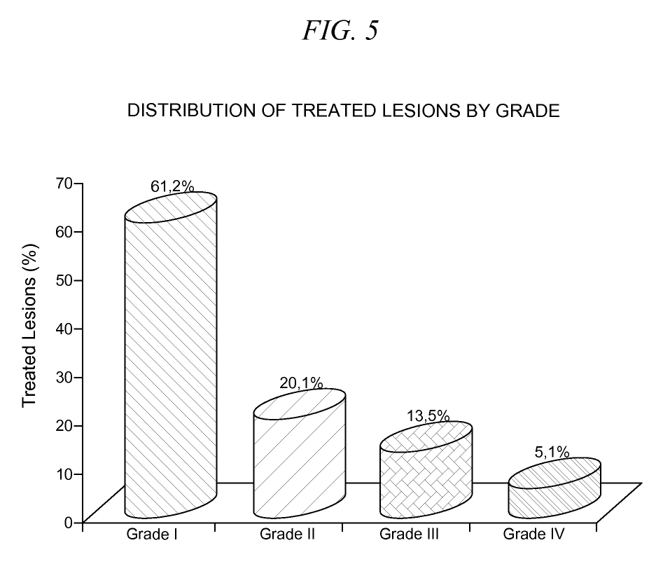

FIG. 6 shows by a table graphic the efficiency in the scar healing of treated lesions pressure ulcers;

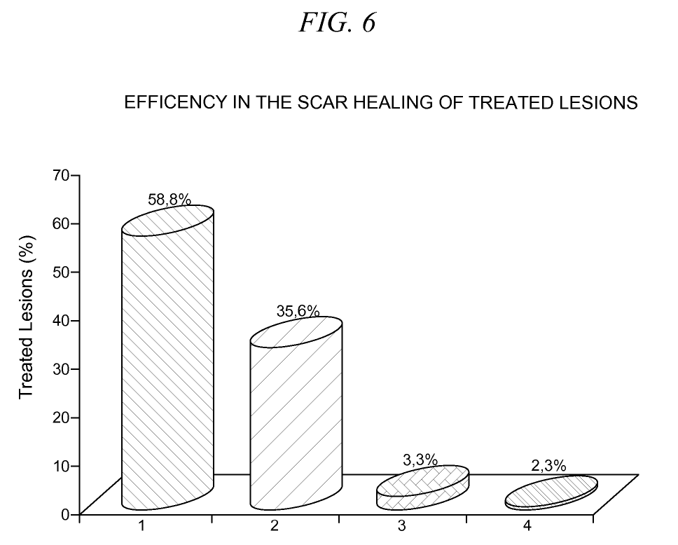

FIG. 7 shows another table graphic of treatment efficiency in connexion with the grade of treated lesion; and

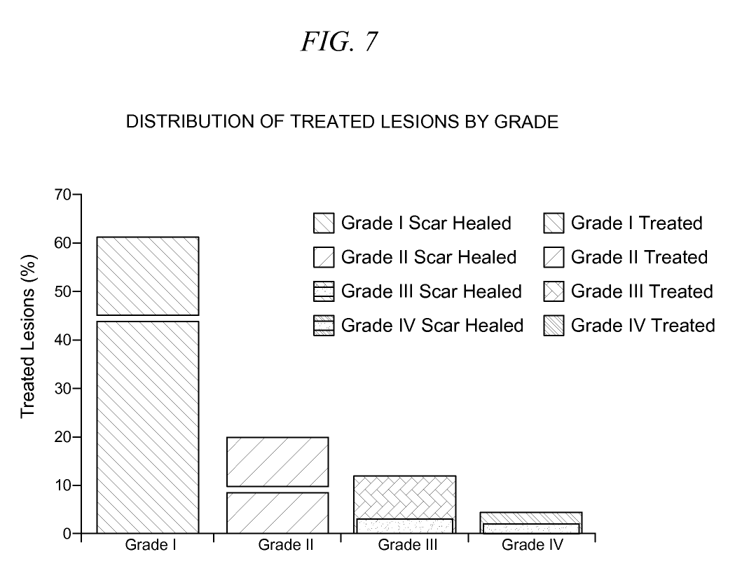

FIG. 8 shows a table graphic of treatment efficiency of varicose ulcers within a period that ranges from 30 to 210 days and as well the efficiency in connection with the grades of classification.

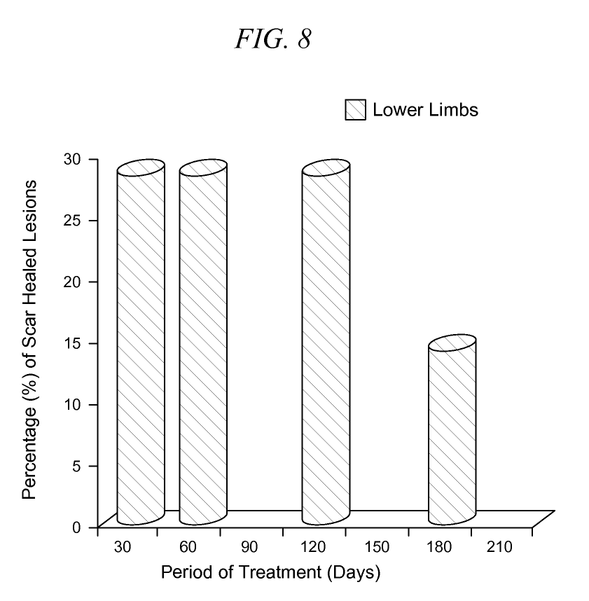

### DETAILED DESCRIPTION OF THE INVENTION

The present invention refers to Phytotherapeutic Pharmaceutical Form with Scar Healing and Microbial Properties and Other Activities of Medical and Veterinary Interest for the Treatment of Ulcers (that either obstruct or decrease the blood irrigation) and Infections; Its Process of Preparation and its Applications and more particularly the present invention disclases a phytotherapeutical formulation applied in the treatment, acceleration of scar healing of lesions and revitalization of the damaged tissue in humans and animals through the topic administration of bioactives derived from plants known as Barbatiman of the gender Stryphnodendron and especimens polyphyllum and adstringens, such a formulation that starts up and stimulate the normal synthesis of colagen with an effective action of tissue recovery and regeneration and antimicrobial action on multiresistent field species colonizers of obstructing lesions (pressure and/or varicose), or still lesions provoked by surgery, ulcerations due to burns and infections located in the lesions.

The phytotherapeutical intermediary preparations of Barbatiman were carried out following determined steps and described next obtaining the following physical-chemical characteristics:

Preparation of Stryphnodendron's Dyes:

Dyes are prepared with the ground barks of the plant, soaked for 15 days in a mixture of cereals alcohol in water (2:1) with determined volume. After soaking, mixtures are filtered and the obtained volume is completed for the sufficient quantity.

Preparation of Concentrated Stryphnodendron's Dyes:

Dyes are concentrated in a rotoelevador under vacuum at a maximum temperature of 600 Celsius, being designed by the solutions' concentrations to levels of 50%, 25% and lo%, respectively as solutions A, B and C. The final aspect of dyes comprises a liquid of dark brown red and strawberry red color when diluted has no peculiar odor and its taste is of binding sensation. Ph is 100%:4.0-4.5 and the contents of total phenols is of 1.70-1.75 g/′100 mL.

Dry Extract of Stryphnodendron:

The dry extracts of Stryphnodendron are obtained from the drought of dyes previously separated placed in open recipients which provide a better evaporation of solvent, left for 5 days in a proofer with air forced circulation at a temperature of 50 degrees. Final aspect comprises a dark brown red color powder and as well of strawberry red color when dissolved in an acquous vehicle, has no peculiar odor and its taste is binding sensation. Total phenols content: 60.0-65.0 gl 100 g.

The formulation developed for the preparation of the phytotherapeutic product contains the dry extract from the Stryphnodendron's bark dye and the assistant substances Nipagin (conservative), glicerin (viscosity agent) monosodium phosphate (pH correction) , the following formulation being defined:

| | |
|---|---|
| Stryphnodendron's dry extract | from 2% to 5% of total phenols |
| | and 1.8% to 40% of tannins; |
| Glicerin | 10%; |
| Nipagin | 0.2% |
| Phosphate Tissue | q.s. pH 5.5-6.0; and |
| Purified Water | 100.0 mL |

In an ideal concentration the formulation determines that the Stryphnodendron's quantity of dry extract should be of 4.4 to 5.0 gl 100 ml to generate a concentration of 60.0 to 65.0 gl 100 g of total phenols, that is, 3% of total phenols in the formulated products.

The studied chemical portions were the proantocianidinas , prorobinetinidinas, flavan-3-01 e prodelfinidinal 1 being the anti-inflammatory activity of Stryphnodendron attributed to the presence of proantocianidinas.

The topic composition may use vehicles like gels, sprays, creams, ointment and any other that will maintain the wound with certain moisture thus favoring the activity of the innovated phytoteutical medicine.

The manufacturing process of the above-referenced formulation will undergo the following steps:

Manufacturing Process:

1—Weighing the determined quantities of Stryphnodendron's dry extract, glicerine, nipagin and measuring the quantity of purified water;
2—Placing the Stryphnodendron's dry extract and the glicerin In a tank that holds an adequate mixer;
3—Adding part of distilled water and start up stirring;
4—Adding the nipagin in a part of tepid water;
5—Adding the dissolved nipagin to the main drum of mixture under agitation;
6—Homogenizing for sufficient time till complete dissolution;
7—Adding the remaining volume of water and heating the moisture up to ebolution for a 10 minutes; Then cooling the moisture;
8—Adding the monosodic phosphate under agitation to set up pH around 5.5-6.0;
9—After said procedure, to complete with distilled water the volume of the produced amount in order to compensate for losses due to evaporation;
10—Beginning the filtration with adequate elements and providing samples for analysis at the quality control; and
11—After release, starting up the accommodation of product in the corresponding packages.

The, phytotherapeutical formulation for scar healing of topic use now innovated might be used, in an alternate way, in association with physiotherapeutical apparatuses as the the ultrasound (US) and low intensity lasers functioning as an accelerator for treatment in the tissue recovery .

Despite being the invention disclosed in details it is important to understand that the same does not limit its application to the details and steps herein described. The invention is able of being used into other applications and of being carried out or executed in a variety of ways. It should be understood that the terminology employed herein is for a describing purpose and not for a limitation.

What is claimed:

1. Composition for topic use containing extract from the plant of gender stryphnodendron: it's preparation process and it's application, phytoterapeutic composition for topic use with the purpose of recovery and tissue regeneration of cutaneous wounds, in humans or animais, as those, for instance, provoked by ulcerations of the type that stops or decrease blood irrigation (pressure or varix ones), provoked by postsurgeries, by irradiation, by laceration, burns or infections, featured to comprise a phytotherapeutic composition for topic use whose active (principles) is derived from the portiioning of plant of gender Stryphnodendron with high content of total phenols and tannins, following a separation of chemical portions, preparation of dyes and susequent process of production of a mixture of dry dyes in a defined and adequate porcentage with assisting agents, taking the topic pharmaceutical forms for the use in the treatment of cutaneous wounds in accordance with clinically evaluated and aproved method.

2. Composition for topic use that contains extract of the plant of the gender Stryphnodendron featured for holding contents of 1%-6% of total phenols.

3. Composition in accordance with claim 1 featured for holding contents of 2% to 5% of total phenols.

4. Composition in accordance with claim 2 featured for holding contents of 2.5% to 3.5% of total phenols.

5. Composition in accordance with claims 1 to 3 featured for the contents of total phenols be comprised by 80% to 90% of tannins.

6. Composition in accordance with claims 1 to 4 featured for being the contents of total phenols essencially constituted of phenolic compounds extracted from plants of gender Stryphnodendron.

7. Composition in accordance with claims 1 to 5 featured for being the contents of total phenols essencially constituted of phenolic compounds extracted from the barks of plants of gender Stryphnodendron po/yphy//um or Stryphnodendron adstringens.

8. Composition in accordance with claim 6 featured for being the contents of total phenols essentially constituted of phenolic compounds extracted from the barks of plants of gender Stryphnodendron po/yphy//um.

9. Composition in accordance with claim 6 featured for being the contents of total phenols essentially constituted of phenolic compounds extracted from the barks of plants of gender Stryphnodendron adstringens.

10. Composition in accordance with claims 1 to 8 featured for holding extract of the plant of gender Stryphnodendron obtained from extraction with water, solvents that can be mixed with water or mixtures of them.

11. Process of prodyction of a topic composition that contains extract of plant of the gender Stryphnodendron featured for adding phenolic compounds for a final content of 1% to 6% of total phenols.

12. Process in accordance with claim 10 featured for adding phenolic compounds for a final content of 2% to 5% of total phenols.

13. Process in accordance with claim 11 featured for adding phenolic compounds for a final content of 2.5% to 3.5% of total phenols.

14. Process in accordance with claims 10 to 12 featured for the contents of total phenols be comprised of 80% to 90% of tannins.

15. Process in accordance with claims 10 to 13 featured for phenolic compounds being derived essentially from plants of the gender Stryphnodendron

16. Process in accordance with claims 10 to 14 featured for phenolic compounds being derived essentially from barks of plants of the gender Stryphnodendron po/yphy//um or Stryphnodendron adstringens.

17. Process in accordance with claim 15 eatured for phenolic compounds being derived essentially from barks of plants of the gender Stryphnodendron.

18. Process in accordance with claim 15 featured for phenolic compounds being derived essentially from barks of plant of the gender Stryphnodendron po/yphy//um adstringens.

19. Process in accordance with the claims 10 to 17 featured for holding extract of the plant of gender Stryphnodendron obtained by extraction with water, solvents that can be mixed with water or mixtures therefrom.

20. Use of the extract of the plant of gender Stryphnodendron for preparation of composition for the treatment of cutaneous wounds featured for holding contents of total phenols of 1% to 6%.

21. Use in accordance with claim 19 featured for the preparation of a composition with a contents of 2% to 5% of total phenols.

22. Use in accordance with claim 19 featured for the preparation of a composition with a content of 2.5% to 3.5% of total phenols.

23. Use in accordance with claims 19 to 21 featured for the contents of total phenols be comprised (or compound???) by 80% to 90% of tannins.

24. Use in accordance with claims 19 to 22 featured for phenolic compounds being essentially derived from barks of the plant of gender Stryphnodendron.

25. Use in accordance with claims 19 to 23 featured for phenolic compounds being essentially derived from barks of the plant of species Stryphnodendron polyphy//um or Stryphnodendron adstringens.

26. Use in accordance with claims 19 to 24 featured for the phenolic compounds being essentially derived from barks of the plant of the species Stryphnodendron po/yphy//um.

27. Use in accordance with claims 19 to 24 featured for the phenolic compounds being essentially derived from barks of the plant of the species Stryphnodendron adstringens.

28. Use in accordance with the claims 19 to 26 featured for holding extract of the plant of gender Stryphnodendron obtained from extraction with water, solvents that can be mixed with water or mixtures therefrom.

29. Method of treatment of cutaneous wounds with a topic composition holding extract of the plant of gender Stryphnodendron featured for the composition holding content of 1% to 6% of total phenols.

30. Method in accordance with claim 28 featured for the composition holding a content of 2% to 5% of total phenols.

31. Method in accordance with claim 29 featured for the composition holding a content of 2.5% to 3.5% of total phenols.

32. Method in accordance with claims 28 and 30 featured for the contents of total phenols be comprised (compound) by 80% to 90% of tannins.

33. Method in accordance with claims 28 to 31 featured phenolic compounds being essentially delivered from barks of plants of gender Stryphnodendron.

34. Method in accordance with claims 28 to 32 featured for containing extracts of the barks of plants of species Stryphnodendron polyphy//um or Stryphnodendron adsfringens.

35. Method in accordance with claims 28 to 33 featured for containing extracts of the barks of plants of species Stryphnodendron po/yphy//um.

36. Method in accordance with claims 28 to 33 featured for containing extracts of the barks of plants of species Stryphnodendron adsfringens.

37. Method in accordance with claims 28 to 35 featured for containing extracts of the plant of gender Stryphnodendron obtained by extraction with water, solvents that can be mixed with water and mixtures therefrom.
